(19) United States
(12) Patent Application Publication
(10) Pub. No.: US 2016/0202264 A1
(43) Pub. Date: Jul. 14, 2016

(54) A high-throughput assay to identify molecules that modulate Rb-E2F binding

(71) Applicant: Seth M. RUBIN, Santa Cruz, CA (US)

(72) Inventor: Seth M Rubin, Santa Cruz, CA (US)

(73) Assignee: The Regents of the University of California, Oakland, CA (US)

(21) Appl. No.: 14/913,550

(22) PCT Filed: Aug. 22, 2014
(86) PCT No.: PCT/US14/52407

§ 371 Date: Feb. 22, 2016

Related U.S. Application Data
(60) Provisional application No. 61/868,889, filed on Aug. 22, 2013.

(51) Int. Cl. G01N 33/68; A61K 31/53

(52) U.S. Cl. CPC G01N 33/68; A61K 31/53; G01N 2500/20; G01N 2500/02; G01N 2333/4704; G01N 2333/4703

(57) ABSTRACT

A high-throughput fluorescence polarization assay to screen molecules for their modulation of Rb-E2F binding affinity.

### RELATIONSHIP TO OTHER APPLICATIONS

The present application claims priority to and the benefit of U.S. provisional application No. 61/868,889 filed 22 Aug. 2013 and titled A High-throughput Assay to Identify Molecules that Modulate Rb-E2F Binding. This application is hereby incorporated by reference for all purposes.

### STATEMENT OF SUPPORT

This invention was made with government support under National Cancer Institute Grant No. ROI CA132685. The government has certain rights in the invention.

### FIELD OF THE INVENTION

The invention encompasses methods for identifying anti-neoplastic compounds.

### BACKGROUND

The Rb pathway connects proliferative growth factor signals to the cell cycle machinery that drives cell division, and therefore it is found deregulated in a large number of tumors, which divide even in the absence of proper signals (Dick and Rubin, 2013). Rb prevents cells from dividing until it is inactivated by Cyclin-dependent kinase (Cdk) phosphorylation. Researchers have sought small molecule inhibitors of Cdks in order to arrest dividing cancer cells; however, obstacles in achieving kinase specificity and pathway redundancy have prevented Cdk drugs from successfully navigating though clinical trials (Stone et al., 2012). More recently, Rb inhibition has been linked with self-renewal of embryonic stem cells, suggesting future utility of selective Rb inactivation in regenerative medicine (Sage, 2012). Despite these motivations for controlling Rb pathway activity, there exist no small molecules that modulate Rb function through direct interaction with the Rb protein. In addition to obvious ultimate clinical applications, there exists tremendous utility in such compounds as experimental tools to probe cellular function and regulation of Rb in vivo. Rb negatively regulates cell division by binding and inhibiting E2F transcription factors (Dick and Rubin, 2013).

The inventors hypothesize that Rb activity can be stimulated by using small molecules to inhibit Rb-E2F dissociation, which occurs upon Cdk phosphorylation (FIG. 1).

The inventors' recently elucidated crystal structure of inactive Rb reveals that phosphorylation induces docking of the Rb N-terminal domain (RbN) to the E2F-binding “pocket” domain (Burke et al., 2012). The interdomain association allosterically rearranges the E2F binding site in the pocket to destabilize transcription factor binding. Preventing this RbN-pocket association will trap the growth suppressive Rb-E2F complex despite phosphorylation of Rb (FIG. 1) and arrest proliferating cells. In addition, molecules that bind Rb and inhibit E2F binding may have useful applications in regenerative medicine and other contexts in which stimulating proliferation is needed.

### BRIEF DESCRIPTION OF THE INVENTION

The invention encompasses a method for performing a high-throughput fluorescence polarization assay for simultaneously screening test compounds for their ability to either stabilize or inhibit binding between a Retinoblastoma tumor suppressor protein (Rb) and an E2F transcription factor (EF2), the method comprising (i) providing one or more test compounds; (ii) synthesizing a peptide corresponding to the E2F transactivation domain, wherein the synthesized peptide comprises a fluorescent dye moiety; (iii) mixing said peptide, in the presence of said one or more test compounds, with one or more Rb constructs which bind the peptide and alter the polarization of fluorescence; (iv) and measuring the fluorescence polarization (FP) ratio, wherein the FP ratio indicates the measure of binding affinity between Rb and E2F. A stabilizing compound will raise the FP ratio and an inhibiting compound will lower the FP ratio. Phosphorylated Rb proteins are used to screen for activators of Rb-E2F binding and unphosphorylated Rb proteins are used to screen for inhibitors of Rb-E2F binding.

In some embodiments the E2F transactivation domain comprises E2F2 amino acids 409-428. The domain may comprise more amino acids but will ideally comprise at least this portion.

The fluorescent dye may be any dye such as a tetramethylrhodamine dye (TMR). The fluorescent dye moiety may be located anywhere in the protein, such as at its N-terminus or C-terminus or between the termini.

FP is calculated as follows: FP=1000*(S−G*P)/(S+G*P), where S is intensity of fluorescence parallel to excitation plane, P is intensity perpendicular to excitation plane, and G is G-factor.

The method may further comprise providing a control compound (such as DMSO) in place of the test compound, and measuring the FP ratio using said control compound, and setting this FP ratio as the control level against which is measured other FP ratios produced using test compounds.

An FP ratio more than two (or for example more than 3) standard deviations above or below the mean FP ratio for the control is considered to indicate the presence of a test compound that either stabilizes or inhibits binding between a Retinoblastoma tumor suppressor protein (Rb) and an E2F transcription factor (EF2).

Additionally, the invention encompasses a high-throughput fluorescence polarization assay to screen molecules for their modulation of Rb-E2F binding affinity.

The exemplary assay disclosed comprises a peptide corresponding to the E2F transactivation domain (E2F2 amino acids 409-428) was synthesized with a tetramethylrhodamine dye (TMR) at its N-terminus (E2F^{TMR}). This peptide can then be mixed with various Rb constructs, which bind the peptide and change the polarization of the TMR fluorescence. For initial experiments, the inventors have used an Rb construct Rb^{NP}, which contains the Rb N-terminal domain and pocket domain but lacks the internal loops in each domain (residues 53-787, Δ245-267, μΔ582-642).

The invention also encompasses newly identified small molecules that modulate Rb function through direct interaction with the Rb protein, for example see FIG. 4.

### BRIEF DESCRIPTION OF THE FIGURES

FIG. 1: Strategy for prevention of Rb inactivation by phosphorylation.

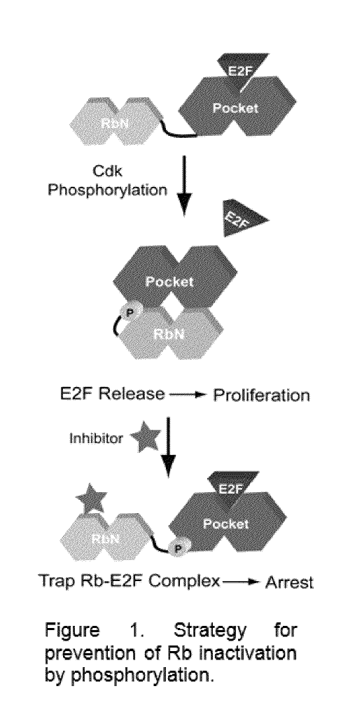

FIG. 2: Titration of RbNP into E2FTMR. The FP ratio increases as the E2F peptide is bound by Rb protein. Phosphorylation of Rb decreases the affinity, and the E7 peptide mitigates the effect of phosphorylation

FIG. 3: Sample data from the primary screen. FP ratio is plotted for each well, which that contains phosphorylated RbNP and compounds (black dots), unphosphorylated RbNP (green triangles, positive control), or free E2FTMR alone (blue triangles). The pink line shows the average FP ratio for phosphorylated RbNP (negative control). Labelled black dots are hits.

FIG. 4: Compounds sharing a common core scaffold based on 1,6-dimethylpyrimido[5,4-e][1,2,4]triazene-5,7(1H,6H)-dione.

### GENERAL REPRESENTATIONS CONCERNING THE DISCLOSURE

All scientific papers, publications, patent documents and other disclosures mentioned herein are hereby incorporated by reference for all purposes.

The embodiments disclosed in this specification are exemplary and do not limit the invention. Other embodiments can be utilized and changes can be made. As used in this specification, the singular forms “a”, “an”, and “the” include plural reference unless the context clearly dictates otherwise. Thus, for example, a reference to “a part” includes a plurality of such parts, and so forth. The term “comprises” and grammatical equivalents thereof are used in this specification to mean that, in addition to the features specifically identified, other features are optionally present. Where reference is made in this specification to a method comprising two or more defined steps, the defined steps can be carried out in any order or simultaneously (except where the context excludes that possibility), and the method can optionally include one or more other steps which are carried out before any of the defined steps, between two of the defined steps, or after all the defined steps (except where the context excludes that possibility). Where reference is made herein to “first” and “second” features, this is generally done for identification purposes; unless the context requires otherwise, the first and second features can be the same or different, and reference to a first feature does not mean that a second feature is necessarily present (though it may be present). Where reference is made herein to “a” or “an” feature, this includes the possibility that there are two or more such features. This specification incorporates by reference all documents referred to herein and all documents filed concurrently with this specification or filed previously in connection with this application, including but not limited to such documents which are open to public inspection with this specification.

The term “derivative” or “derivative compound” refers to a compound having a chemical structure that contains a common core chemical structure as a parent or reference compound, but differs by having at least one structural difference, e.g., by having one or more substituents added and/or removed and/or substituted, and/or by having one or more atoms substituted with different atoms. Unless clearly indicated to the contrary, the term “derivative” does not mean that the derivative is synthesized using the parent compound as a starting material or as an intermediate, although in some cases, the derivative may be synthesized from the parent.

The term “fragment” refers to a part of a larger whole, for example a fragment of a molecule may be any dissociated part of that molecule, regardless of size.

The term “specie” or “group” when used to describe an “R” group in a chemical formula, is used to mean any chemical compound, sub-compound or substituent that may chemically interact with (covalently, ionically or by Van der Waal's forces) another molecule or group such as shown on a chemical formula.

When a “terminus” or “terminal group” is discussed as having a substituent, side-chain, group or moiety attached, that substituent, side-chain, group or moiety may equally be present at one or more termini or at side locations along the length of the molecule.

### DETAILED DESCRIPTION OF THE INVENTION

Fluorescence Polarization Assay of Rb-E2F Binding.

The inventors have designed and successfully implemented a high-throughput fluorescence polarization assay to screen molecules for their modulation of Rb-E2F binding affinity. In the current exemplary format, a peptide corresponding to the E2F transactivation domain (E2F2 amino acids 409-428) was synthesized with a tetramethylrhodamine dye (TMR) at its N-terminus (E2FTMR). This peptide can then be mixed with various Rb constructs, which bind the peptide and change the polarization of the TMR fluorescence. For initial experiments, the investigators have used an Rb construct RbNP, which contains the Rb N-terminal domain and pocket domain but lacks the internal loops in each domain (residues 53-787, Δ245-267, Δ582-642).

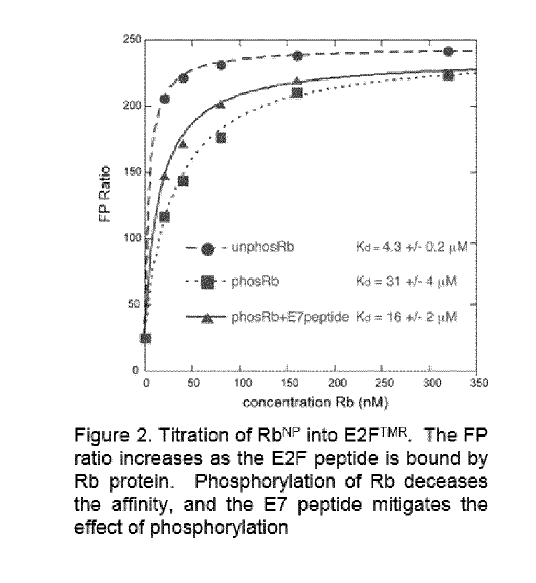

As an initial demonstration of the assay, the investigators titrated both unphosphorylated and phosphorylated RbNP into 10 nM E2FTMR and measured the change in fluorescence polarization ratio (FP=1000*(S−G*P)/(S+G*P), where S is intensity of fluorescence parallel to excitation plane, P is intensity perpendicular to excitation plane, and G is G-factor). As seen in FIG. 2, the FP ratio changes from ˜20 for free E2FTMR to ˜225 for E2FTMR fully bound with RbNP. The affinity of E2FTMR for unphosphorylated Rb is approximately 7-fold tighter than its affinity for phosphorylated RbNP. In the presence of 10 μM synthetic peptide that contains the ‘L×C×E’ sequence from the E7 oncoprotein, phosphorylated Rb binds E2F with 2-fold higher affinity. This peptide, which has a similar effect in calorimetry measurements of affinity, binds near the RbN-pocket interface, and the investigators predict it inhibits interdomain docking.

A tremendous advantage of this FP assay is the ability to test simultaneously for compounds that either stabilize or inhibit E2F binding. Both types of molecules may have laboratory and clinical applications. The investigators screened in 384-well format with 10 nM phosphorylated RbNP. The FP ratio for E2FTMR in the presence of 10 nM phosphorylated RbNP is ˜60. A stabilizing compound will raise this ratio towards its value for unphosphorylated RbNP (˜165 at 10 nM), while an inhibiting compound will lower the FP ratio towards that of unbound E2FTMR (˜20).

Preliminary Screen Results

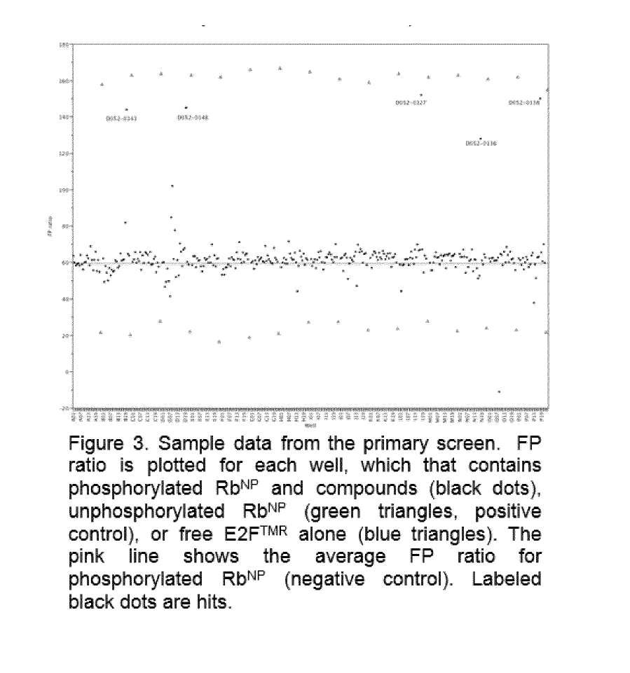

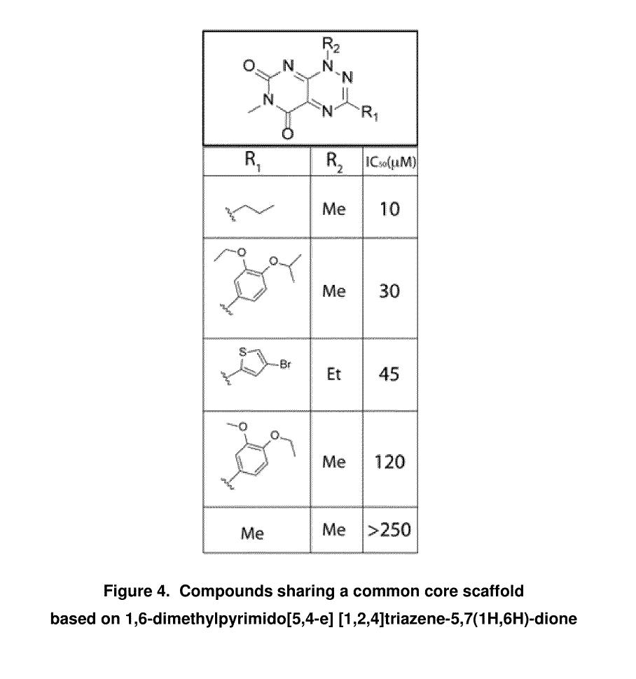

The investigators tested ˜21,000 compounds from the ChemDiv library in the UCSC Chemical Screening Center at a concentration of 50 μM. Data from one 384-well plate in the screen are shown in FIG. 3. In this primary screen, the assay had an average z′=0.79. 74 compounds resulted in an FP ratio more than three standard deviations above or below the mean FP ratio for phosphorylated RbNP alone (negative control). These compounds were than used in a secondary assay in which phosphorylated RbNP was titrated in the presence of E2FTMR and compound. The investigators found that 7 compounds, all of which enhanced E2F binding to phosphorylated RbNP, significantly changed the Kd. The remaining compounds did not change the Kd in the secondary titration assay, or they affected the FP ratio of E2FTMR in the absence of RbNP. 5 of the 7 hits contain a common core scaffold based on 1,6-dimethylpyrimido[5,4-e][1,2,4]triazene-5,7(1H,6H)-dione. The investigators were able to measure IC50 values for these compounds, which gives us preliminary structure-activity relationship information (See Table FIG. 4).

### REFERENCES

Burke, J. R., Hura, G. L., and Rubin, S. M. (2012). Structures of inactive retinoblastoma protein reveal multiple mechanisms for cell cycle control. Genes Dev 26, 1156-1166.
Dick, F. A., and Rubin, S. M. (2013). Molecular mechanisms underlying RB protein function. Nat Rev Mol Cell Biol 14, 297-306.
Sage, J. (2012). The retinoblastoma tumor suppressor and stem cell biology. Genes Dev 26, 1409-1420.
Stone, A., Sutherland, R. L., and Musgrove, E. A. (2012). Inhibitors of cell cycle kinases: recent advances and future prospects as cancer therapeutics. Crit Rev Oncog 17, 175-198

1. A method for performing a high-throughput fluorescence polarization assay for simultaneously screening test compounds for their ability to either stabilize or inhibit binding between a Retinoblastoma tumor suppressor protein (Rb) and an E2F transcription factor (EF2), the method comprising (i) providing one or more test compounds; (ii) synthesizing a peptide corresponding to the E2F transactivation domain, wherein the synthesized peptide comprises a fluorescent dye moiety; (iii) mixing said peptide, in the presence of said one or more test compounds, with one or more Rb constructs which bind the peptide and alter the polarization of fluorescence; (iv) and measuring the fluorescence polarization (FP) ratio, wherein the FP ratio indicates the measure of binding affinity between Rb and E2F, wherein a stabilizing compound will raise the FP ratio towards its value for unphosphorylated RbNP, while an inhibiting compound will lower the FP ratio towards that of unbound E2FTMR.

2. The method of claim 1 wherein said E2F transactivation domain comprises E2F2 amino acids 409-428.

3. The method of claim 1 wherein said fluorescent dye is a tetramethylrhodamine dye (TMR).

4. The method of claim 1 where the FP is calculated as follows: FP=1000*(S−G*P)/(S+G*P), where S is intensity of fluorescence parallel to excitation plane, P is intensity perpendicular to excitation plane, and G is G-factor.

5. The method of claim 1 wherein the synthesized peptide comprises a fluorescent dye moiety at its N-terminus.

6. The method of claim 1 wherein a stabilizing compound will raise the FP ratio and an inhibiting compound will lower the FP ratio.

7. The method of claim 1 wherein phosphorylated Rb proteins are used to screen for activators of Rb-E2F binding.

8. The method of claim 1 wherein un-phosphorylated Rb proteins are used to screen for inhibitors of Rb-E2F binding.

9. The method of claim 1 further comprising providing a control compound in place of said test compound, and measuring the FP ratio using said control compound, and setting this FP ratio as the control level against which is measured other FP ratios produced using test compounds.

10. The method of claim 9 wherein an FP ratio more than two standard deviations above or below the mean FP ratio for the control is considered to indicate the presence of a test compound that either stabilizes or inhibits binding between Rb and EF2.

11. The method of claim 1 wherein the one or more Rb constructs comprise the Rb N-terminal domain and pocket domain but lacks one or more internal loops in each domain.

12. The method of claim 11 wherein the Rb constructs lack the internal loops in each domain comprising residues 53-787, Δ245-267, Δ582-642.

13. The method of claim 11 wherein the synthesized peptide corresponds to the inactive form of the phosphorylated Rb protein.

14. The method of claim 11 wherein the synthesized peptide further comprises a dye moiety at its N-terminus.

15. The method of claim 14 wherein the dye moiety at the N-terminus comprises a tetramethylrhodamine (TMR) dye.

16. The method of claim 11 wherein the test compound is a compound is selected from the group consisting of:

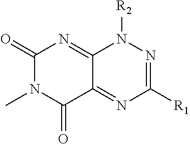

| R₁ | R₂ | IC₅₀ (μM) |
|---|---|---|
| 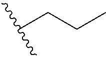 [SMILES: *CCC] | Me | 10 |
| 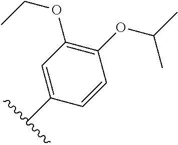 [SMILES: *c1ccc(OC(C)C)c(OCC)c1] | Me | 30 |
| 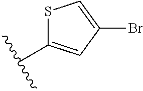 [SMILES: *c1cc(Br)cs1] | Et | 45 |
| 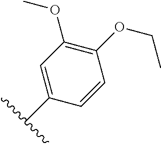 [SMILES: *c1ccc(OCC)c(OC)c1] | Me | 120 |
| Me | Me | >250 |

17. The method of claim 11 wherein the test compound is 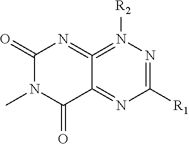
wherein 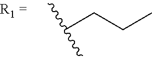
and herein R₂=Me.

18. The method of claim 11 wherein the test compound is 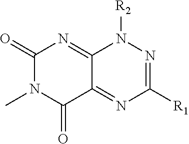
wherein 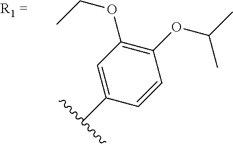 [SMILES: *c1ccc(OC(C)C)c(OCC)c1]
and wherein R₂=Me.

19. The method of claim 11 wherein the test compound is 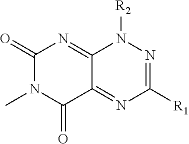
wherein 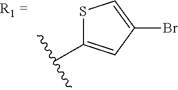 [SMILES: *c1cc(Br)cs1]
and wherein R₂=Et.

20. The method of claim 11 wherein the test compound is 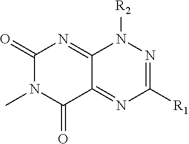
wherein 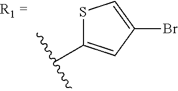 [SMILES: *c1cc(Br)cs1]
and wherein R₂=Me.

21. A method for altering binding between a Retinoblastoma tumor suppressor protein (Rb) and an E2F transcription factor (EF2), the method comprising (i) providing a binding-altering compound; (ii) providing a Retinoblastoma tumor suppressor protein (Rb); (iii) providing an E2F transcription factor (EF2); (iv) mixing together said components (i), (ii), and (iii).

22. The method of claim 21 wherein the binding-altering compound is 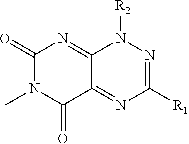
wherein 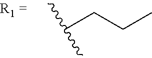
and herein R₂=Me.

23. The method of claim 21 wherein the binding-altering compound is 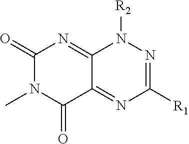
wherein 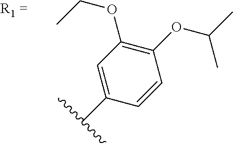 [SMILES: *c1ccc(OC(C)C)c(OCC)c1]
and wherein R₂=Me.

24. The method of claim 21 wherein the binding-altering compound is 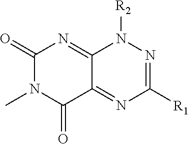
wherein 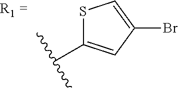 [SMILES: *c1cc(Br)cs1]
and wherein R₂=Et.

25. The method of claim 21 wherein the binding-altering compound is 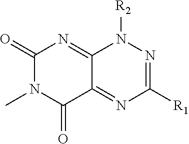
wherein 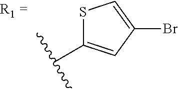 [SMILES: *c1cc(Br)cs1]
and wherein R₂=Me.
